(19) United States
(12) Patent Application Publication
(10) Pub. No.: US 2024/0191184 A1
(43) Pub. Date: Jun. 13, 2024

(54) CELL SYSTEM AND METHODS OF USE

(71) Applicant: MelliCell, Inc., Newton, MA (US)

(72) Inventors: Aaron L. GLIEBERMAN, Somerville, MA (US); Benjamin D. POPE, Watertown, MA (US); David C. BOUCK, Arlington, MA (US)

(21) Appl. No.: 18/286,077

(22) PCT Filed: Apr. 8, 2022
(86) PCT No.: PCT/US2022/024061

§ 371 Date: Oct. 6, 2023

Related U.S. Application Data
(60) Provisional application No. 63/172,990, filed on Apr. 9, 2021.

(51) Int. Cl. C12N 5/00; C12N 5/077; G01N 33/50

(52) U.S. Cl. CPC C12N 5/0068; C12N 5/0653; G01N 33/5044; C12N 2533/30; C12N 2533/54; C12N 2539/10

(57) ABSTRACT

In some embodiments, the present disclosure provides cell systems that include arrangements of cells maintained by various separation systems and/or support layers for culture and differentiation of cells as well as related methods of use and preparation.

### CROSS REFERENCE TO RELATED APPLICATIONS

This application claims priority to U.S. Provisional Patent Application No. 63/172,990 entitled “Cell System and Methods Of Use” filed on 9 Apr. 2021, the contents of which are herein incorporated by reference in their entirety.

### BACKGROUND

Discovery and development of therapeutic agents is challenging due to difficulties with developing sufficient systems and models to assess their function and efficacy. This is especially true of therapeutic agents targeting adipose tissue. High density cell cultures can result in geometric constraints which may yield a rounded cell shape that destabilizes the cytoskeleton in preadipocytes and predisposes them to become adipocytes. Conversely, when preadipocytes are allowed to spread, adipocyte differentiation is inhibited in favor of osteocyte formation. While starting with high density cultures of preadipocytes is efficient for differentiating adipocytes from immature cells, it dramatically slows their size growth (i.e., hypertrophy) and maturation.

There remains a need in the art for cell systems, including cell systems that include mature adipocytes, for discovery, development, and testing of therapeutic agents useful for treating obesity and related metabolic disorders. There also remains a need in the art for cell systems for preparing and growing cells, including mature adipocytes, for various uses. The present disclosure addresses this need with cell systems and methods of use described herein.

### SUMMARY

In some embodiments, the present disclosure provides a cell separation system comprising a substrate; and a cell separation feature in or on the substrate for spatially separating individual cells. The cell separation features may provide a cell spacing between adjacent cells of from about 5 μm to about 500 μm. The substrate may comprise a cell culture surface. The cell culture surface may comprise one or more of a cell culture dish, a cell culture well, a plastic, a resin, elastomer, metal, and/or glass. The cell separation feature may include a cell separation structure, and/or a coating layer for promoting or discouraging cell attachment. The cell separation structure may comprise a pocket in the cell culture surface, and the pocket may be a microwell. The cell separation structure may comprise a projection from the cell culture surface, and the projection may be a pillar. The cell separation feature may provide a cell attachment surface having a diameter of from about from about 25 μm to about 50 μm and/or a cross sectional area of area of from about 400 μm² to about 200 μm². The cell attachment surface may be substantially flat. The cell attachment surface may be circular, oblong, rectangular, or irregular in shape. The cell separation features may be a pocket, and the cell attachment surface may comprise a bottom surface of the pocket. The cell separation feature may include a projection, and the cell attachment surface may comprise a top surface of the projection. The cell separation feature may be formed by one or more of etching, micromolding, milling and 3D printing. The cell separation structure may comprise an array of cell separation structures in or on the substrate. The cell separation system may further comprise a coating layer. The cell separation structure may provide a cell attachment surface, and the coating layer may cover the cell attachment surface. The coating layer may comprise a coating protein. The coating protein may comprise an extracellular matrix protein. The extracellular matrix protein may comprise collagen, laminin, fibronectin, gelatin, or a fragment or combination thereof.

In some embodiments, there is provided a cell support member which may include a porous material. The cell support member may maintain a position of a cell on a substrate. The cell support member may be configured for placement over the cell to prevent passage of the cell through the porous material while allowing fluid communication through the porous material. The cell support member may include a cell support body; optionally, a hole through the cell support body. The porous material may include a thermoplastic material. The porous material may be a membrane or a disc. The porous material may comprise a synthetic polymer and/or a biogenic polymer. The synthetic polymer may comprise one or more of a poly(urethane), a poly(siloxane), a poly(ethylene), a poly(vinyl pyrrolidone), a poly(2-hydroxy ethyl methacrylate), a poly(N-vinyl pyrrolidone), a poly(methyl methacrylate), a poly(vinyl alcohol), a poly(acrylic acid), a polyacrylamide, a poly(ethylene-co-vinyl acetate), a poly(ethylene glycol), a poly(methacrylic acid), a polylactides (PLA), a polyglycolide (PGA), a poly(lactide-co-glycolide) (PLGA), a polyanhydride, a polyphosphazene, a polygermane, a polyorthoester, a polyester, a polyamide, a polyolefin, a polycarbonate, a polyaramide, a polyimide, a polycaprolactone (PCL), and a copolymer, derivative, or combination thereof. The biogenic polymer may comprise one or more of a protein, a polypeptide, a polysaccharide, a lipid, a nucleic acid, a glycosaminoglycan, a derivative or a combination thereof. The biogenic polymer may comprise one or more of silk, fibroin, sericin, keratin, alpha-keratin, beta-keratin, alginate, elastin, fibrillin, fibrillin-1, fibrillin-2, fibrillin-3, fibrillin-4, fibrinogen, fibrin, fibronectin, laminin, collagen, collagen I, collagen II, collagen III, collagen IV, collagen V, collagen VI, vimentin, neurofilament, light chain neurofilament (NF-L), medium chain neurofilament (NF-M), heavy chain neurofilament (NF-H), amyloid, alpha-amyloid, beta-amyloid, actin, myosin, titin, gelatin, chitin, hyaluronic acid, D-glucuronic acid, D-N-acetylglucosamine, a derivative or a combination thereof. The porous material may include one or more polymer fibers, which may have a diameter of from about 10 nm to about 5000 nm, or from about 200 nm to about 1000 num. The polymer fibers may comprise PCL. The polymer fibers may comprise gelatin. The polymer fibers may comprise at least about 50 wt % PCL. The polymer fibers may comprise from about 1 to about 49 wt % gelatin. The polymer fibers may comprise about 70 wt % PCL and about 30 wt % gelatin. The porous material may be formed from one or a combination of controlled spinning of nanofibers, lithography, deposition, etching, molding and crystal growth. The porous material may have a porosity of from about 20% to about 50%. The average pore size in the porous material may be from about 1 μm to about 5 μm. The cell support member may be substantially planar. The cell support member may be substantially tubular. The cell support member may have one or more tabs projecting from its outer perimeter for engaging with a cell separation system for aligning and/or anchoring the cell support member to the cell separation system.

In some embodiments, there is provided a cell support layer comprising an array of cell support members, wherein the cell support members are retained by an array body.

In some embodiments, there is provided a cell system comprising a cell separation system and a cell support member configured for engagement with the cell separation structure. In some embodiments, there is provided a cell system comprising an array of cell separation structures; and a cell support layer, wherein the array of cell support members are configured to align with the array of cell separation structures. The cell support members may comprise a substantially planar cell support body configured for insertion into or over the cell separation structure comprising pockets or placement over a projection of the cell separation structure. The cell support members may comprise a substantially tubular cell support body configured for insertion into or over the cell separation structure comprising pockets or placement over a projection of the cell separation structure.

The arrangement of individual cells may comprise a progenitor cell, such as but not limited to a mesenchymal progenitor cell. The arrangement of individual cells may comprise one or more of a pre-adipocyte and a mature adipocyte. The mature adipocyte may comprise a diameter of from about 15 μm to about 400 μm. The distance between adjacent cells may be from about 5 μm to about 500 μm. The cell system may be configured for high throughput analysis of at least one test factor. The cell system may be configured for analysis of multiple conditions relative to the at least one test factor.

In some embodiments, there is provided a placement system for engaging a cell support member with a cell separation system comprising: a punch having at least one punch head with a distal face; an optional alignment system for aligning the punch with the cell support member; an actuating system for engaging the at least one punch head with the cell support member to disengage the cell support member from a cell support layer, and to engage the cell support member with the cell separation system. The punch may comprise a plurality of punch heads configured for alignment with an array of cell support members on the cell support layer, for disengaging multiple cell support members from the cell support layer simultaneously. The at least one punch head may comprise a recess on its distal face for preventing contact between the distal face and one or more holes in the cell support member during disengagement of the cell support member. The actuating system may be actuated manually, automatically or semi-automatically. The actuating system may comprise any one or combination of one or more motors, pulleys and pistons.

In accordance with some embodiments, there is provided a method of forming a cell support member comprising the steps of: providing a sheet of material; cutting at least one hole in the sheet of material; forming a support member body from the sheet of material by cutting an outer perimeter of the support member body around the hole; and applying a porous material over the hole. When cutting the outer perimeter of the support member body, at least a portion of the outer perimeter may be left uncut such that the support member body remains connected to the sheet of material. There may be multiple support members formed in the sheet of material to form an array of support members. The porous material may be applied over one face of the sheet of material, covering the holes and the support member bodies.

In accordance with some embodiments, there is provided a method of preparing a cell system, the method comprising: providing a cell separation system comprising a cell separation structure on a substrate; and associating a cell with an attachment area on the substrate. There may further comprise the step of applying a coating layer to the attachment area. The coating layer may comprise a coating protein. The coating protein may comprise an extracellular matrix protein. The extracellular matrix protein may comprise collagen, laminin, fibronectin, gelatin, or a fragment of combination thereof. Applying the coating layer may comprise microcontact printing, photolabile polymer printing, and/or stamping. The coating layer may be applied to the attachment area by positioning a blocking mask over the substrate, wherein the blocking mask comprises a hole and wherein the blocking mask is positioned to align the hole with the attachment area; applying the coating layer to the blocking mask; and removing the blocking mask to yield application of the coating layer to the attachment area. From about 0.01 μg to about 50 μg of the coating protein may be applied per square centimeter of attachment area. Associating the cell with the attachment area may comprise contacting the coating layer with a solution of cells, allowing the cell to associate with the coating layer, and removing any cells not associated with the coating layer. The solution of cells comprises a cell concentration suitable to provide from about 1 to about 10,000 cells per square millimeter of attachment area. The cell may comprise one or more of a progenitor cell, a mesenchymal progenitor cell, a pre-adipocyte, and a mature adipocyte. There may further comprise the step of covering the cell with a cell support member after the cell is associated with the attachment area. The cell support member may be inserted into or over a pocket of the cell separation system. The cell support member may be placed over a projection of the cell separation system. Covering the cell with the cell support member may comprise providing a cell support layer comprising an array of cell support members retained by an array body; aligning the cell support layer over the cell separation system such that the cell support members are aligned on a vertical plane with respective cell separation structures; and applying a force to the cell support members to disengage them from the array body and engage them with the cell separation structures. The cell support members in the array may be disengaged simultaneously. A placement system may be used to disengage the cell support members.

An alignment system may be used to align the cell support layer over the cell separation system. The alignment system may comprise a frame configured for aligning with structural features on the cell support layer and the cell separation system. The alignment system may comprise one or more projections and one or more corresponding recesses on any one or combination of the cell support layer, the cell separation system, and a separate alignment member.

The cell system may be configured for high throughput analysis of at least one test factor. The cell system may be configured for analysis of multiple conditions relative to the at least one test factor.

In some embodiments, there is provided a method of analyzing a test factor, the method comprising contacting the cell system with the test factor. The method may comprise contacting the cell system with a control factor and analyzing the test factor by comparison to the control factor.

In some embodiments, there is provided a method of screening for, identifying, or testing a therapeutic agent, the method comprising contacting the cell system with the therapeutic agent and measuring at least one outcome associated with the therapeutic agent. There may further comprise contacting the cell system with a control agent, measuring at least one outcome associated with the control agent, and comparing the outcome associated with the control agent with the outcome associated with the therapeutic agent.

In some embodiments, the present disclosure provides a device for assembly of a cell system according to any of those described herein. The device may include a cell support layer that may be positioned above cell separation system. The device may include a reservoir layer positioned above the cell support layer and/or the cell separation system. The device may include a gasket between the reservoir layer and the cell support layer. The device may include a gasket between the cell support layer and a base layer of the device.

### BRIEF DESCRIPTION OF THE DRAWINGS

The foregoing and other objects, features and advantages will be apparent from the following description of particular embodiments of the invention, as illustrated in the accompanying drawings. Various features of the embodiments described herein can be fully appreciated as the same becomes better understood when considered in light of the accompanying drawings. The drawings are not necessarily to scale but are used to illustrate relative placement of various features.

FIG. 1 is a top view of a substantially planar support member body in accordance with some embodiments.

FIG. 2A is a top view of a substantially planar cell support member in accordance with some embodiments.

FIG. 2B is a side view of the cell support member of FIG. 2A.

FIG. 3 is a perspective view of a substantially tubular cell support member in accordance with some embodiments.

FIG. 4A is a side view of a planar support member inserted into a cell substrate, the cell substrate being a well comprising cell separation features and an arrangement of individual cells, in accordance with some embodiments.

FIG. 4B is a side view of a tubular support member inserted into a cell substrate, the cell substrate being a well comprising cell separation features and an arrangement of individual cells, in accordance with some embodiments.

FIG. 5 is a top view of an array of support members comprising planar support members, where one row of the support members have been disengaged from the array body in accordance with some embodiments.

FIG. 6 is a top view of a section of an array of support members comprising planar support members, with some of the support members inserted into wells containing an arrangement of cells and cell separation features in accordance with some embodiments.

FIGS. 7A, 7B, 7C are side views of a method for forming a cell separation system using a stamp comprising projections for forming pockets in a substrate for containing cells in accordance with some embodiments.

FIG. 8 is a top view of an array of support members being formed from a piece of material in accordance with some embodiments.

FIG. 9A is a top perspective view of an alignment frame in accordance with some embodiments.

FIG. 9B is a top view of the alignment frame of FIG. 9A.

FIG. 9C is a bottom view of the alignment frame of FIG. 9A.

FIG. 10 is a side perspective view showing how an array of support members and a cell separation system are aligned together using an alignment frame in accordance with some embodiments.

FIG. 11 is a perspective view of a punch for disengaging cell support members in accordance with some embodiments.

FIG. 12A is a top view of a cell support member body in accordance with some embodiments.

FIGS. 12B, 12C, 12D are end views of punch head distal ends with various shapes and dimensions for disengaging cell support members in accordance with some embodiments.

FIG. 13 is a top perspective view of an array of punches in accordance with some embodiments.

FIG. 14 is a top perspective view of an array of punches formed from modular subarrays.

FIG. 15A is a side perspective view of the configuration of an array of punches, a cell support layer and a cell substrate comprising cell separation features, the array of punches for disengaging support members from the cell support layer and inserting them into wells in the cell substrate over cell separation features in accordance with some embodiments.

FIG. 15B is a side perspective view of the configuration of FIG. 15A after the punches have disengaged and inserted support members into the wells.

FIG. 16A is an alignment and actuating system for aligning punches with a cell support layer and a cell substrate comprising cell separation features to disengage support members and insert them into wells in the cell substrate over cell separation features in accordance with some embodiments.

FIG. 16B is an enlarged view of a section of the alignment and actuating system of FIG. 16A.

### DETAILED DESCRIPTION

### I. Cell Systems

In some embodiments, the present disclosure provides cell systems. As used herein, the term “cell system” refers to any structure, organization, or arrangement of cells used to support, maintain, and/or to utilize the cells for some purpose (e.g., to discover and/or evaluate therapeutics). Cell systems may include arrangements of individual cells. The arrangements may include specific cell patterning or positioning. In some cells systems, cells are arranged with essentially equal distances between adjacent cells in the arrangement. In some cell systems, cells are arranged to include a range of distances between cells or to include a specific pattern of cells.

Cell systems of the present disclosure may include any cell type. In some embodiments, cell systems include cells that respond to or are otherwise affected by spacing between adjacent cells and/or general positioning relative to other cells or other cell system components. In some embodiments, cell systems include progenitor cells, which are immature cells capable of differentiation into more mature cell types. Arrangement of individual progenitor cells in cell systems may influence the rate and outcome of differentiation of such progenitor cells. In some embodiments, cell system cells include preadipocytes, which refer to immature cell types capable of differentiation into adipocytes. Preadipocytes may include mesenchymal progenitor cells and/or adipose stem cells. Mesenchymal progenitor cells include cells capable of differentiation into adipocytes and other mesodermal lineage cells. Arrangement of individual preadipocytes in cell systems may influence the rate and outcome of differentiation (e.g., differentiation to adipocytes) of such cells. In some cell systems, arrangements of individual cells may include mature adipocytes. Such mature adipocytes may be differentiated prior to or after introduction to cell systems. Preadipocytes present in cells systems may include a diameter of from 1 μm to about 100 μm. As a non-limiting example, preadipocytes present in cell systems may include a diameter of from about 10 μm to about 15 μm. Mature adipocytes present in cell systems may include a diameter of from about 15 μm to about 400 μm (e.g., e.g., from about 15 μm to about 60 μm, from about 20 μm to about 60 μm, from about 30 μm to about 70 μm, from about 40 μm to about 80 μm, from about 50 μm to about 90 μm, from about 45 μm to about 100 μm, from about 50 μm to about 110 μm, from about 55 μm to about 120 μm, from about 60 μm to about 130 μm, from about 65 μm to about 140 μm, from about 70 μm to about 150 μm, from about 75 μm to about 160 μm, from about 80 μm to about 170 μm, from about 85 μm to about 180 μm, from about 90 μm to about 190 μm, from about 95 μm to about 200 μm, from about 100 μm to about 210 μm, from about 115 μm to about 220 μm, from about 125 μm to about 240 μm, from about 135 μm to about 260 μm, from about 145 μm to about 280 μm, from about 155 μm to about 300 μm, from about 165 μm to about 320 μm, from about 175 μm to about 340 μm, from about 185 μm to about 360 μm, from about 195 μm to about 380 μm, or from about 200 μm to about 400 μm).

### Cell Separation Systems

In some embodiments, the present disclosure provides cell separation systems for separating individual cells from each other in cell systems. Cell separation systems may include cell separation features in or on a cell system surface or substrate (e.g., cell culture surfaces). Cell separation features may include coating layers and/or structural features. Cell separation features may be configured to influence cell growth to produce cells having desired sizes and shapes.

In some embodiments, the present disclosure provides cell separation systems including arrangements of individual cells that include spacing between adjacent cells of from about 5 μm to about 500 μm and wherein the position of each individual cell in the arrangement may be maintained by one or more cell separation features. The cell separation features may be included in cell system substrates (e.g., cell culture surfaces).

In some embodiments, cell separation features may be arranged as an array in or on cell system surfaces or substrates. The cell separation features may physically separate individual cells in a homogenous pattern or a non-homogenous arrangement. The cell system surface or substrate may include secondary structures. The secondary structures may be pathways connecting separated cells that allow fluid communication between cells for cell system factors to move between cells, while preventing the passage of cells through the pathways. The pathways may be channels that allow for transmission of intercellular signaling factors. The secondary structures may include partitions for separating the array of cell separation structures into subarrays. Within each subarray, there may be pathways connecting separated cells within the subarray.

In some embodiments, the present disclosure provides cell systems including arrangements of individual cells that include a spacing between adjacent cells of from about 5 μm to about 500 μm (e.g., from about 5 μm to about 40 μm, from about 10 μm to about 50 μm, from about 20 μm to about 60 μm, from about 30 μm to about 70 μm, from about 40 μm to about 80 μm, from about 50 μm to about 90 μm, from about 45 μm to about 100 μm, from about 50 μm to about 110 μm, from about 55 μm to about 120 μm, from about 60 μm to about 130 μm, from about 65 μm to about 140 μm, from about 70 μm to about 150 μm, from about 75 μm to about 160 μm, from about 80 μm to about 170 μm, from about 85 μm to about 180 μm, from about 90 μm to about 190 μm, from about 95 μm to about 200 μm, from about 100 μm to about 210 μm, from about 115 μm to about 220 μm, from about 125 μm to about 240 μm, from about 135 μm to about 260 μm, from about 145 μm to about 280 μm, from about 155 μm to about 300 μm, from about 165 μm to about 320 μm, from about 175 μm to about 340 μm, from about 185 μm to about 360 μm, from about 195 μm to about 380 μm, from about 205 μm to about 400 μm, from about 215 μm to about 420 μm, from about 225 μm to about 440 μm, from about 235 μm to about 460 μm, or from about 245 μm to about 500 μm). In some embodiments, the spacing between adjacent cells may be essentially equal. In some embodiments, the spacing between adjacent cells is unequal.

In some embodiments, cell systems include features that promote cell attachment in some areas and discourage or prevent cell attachment in other areas. Cell attachment may be promoted or discouraged by providing microenvironmental cues. Microenvironmental cues may be provided using coating layers. Microenvironmental cues may be provided from the properties of materials used to form cell separation systems. Cell attachment may be discouraged using surfaces that are hydrophobic or have low adhesion properties. This may be accomplished by forming cell separation systems from materials that are naturally hydrophobic or have low adhesion properties, or have been treated to have such properties. Cell attachment may be promoted using coating layers and/or cell support layers. Cell separation features may be used to geometrically constrain cells such that they may have a rounded shape (relative to cells that can spread unimpeded on a cell culture substrate). In some embodiments, geometrical constraints, including cell coating layers and structural features such as pockets and projections, may be configured to limit cells, such as preadipocytes and adipocytes, to have a largest cross-sectional area on a primary attachment surface from about 400 μm² to about 2000 μm². In some embodiments, the cross sectional area may be from about 490 μm² to about 1964 μm².

In some embodiments, geometrical constraints, including cell coating layers and structural features such as pockets and projections, may be configured to limit cells, such as preadipocytes and adipocytes, to have an average diameter on a primary attachment surface from about 5 μm to about 500 μm, e.g. from about 10 μm to about 300 μm, from about 15 μm to about 200 μm, from about 20 μm to about 100 μm, from about 25 μm to about 75 μm and/or from about 25 μm to about 50 μm. Cell separation features may be configured to prevent cells from expanding too much in one plane compared to the other planes to prevent excessive surface tension of the cell. Cells attached to a primary attachment surface, may include at least one dimension (length, width, or height) from about 5 μm to about 500 μm, e.g., from about 10 μm to about 300 μm, from about 15 μm to about 200 μm, from about 20 μm to about 100 μm, from about 25 μm to about 75 μm, from about 25 μm to about 50 μm. In some embodiments, the cells have at least one dimension of about 1 μm, 2 μm, 3 μm, 4 μm, 5 μm, 6 μm, 7 μm, 8 μm, 9 μm, 10 μm, 1 μm, 12 μm, 13 μm, 14 μm, 15 μm, 16 μm, 17 μm, 18 μm, 19 μm, 20 μm, 21 μm, 22 μm, 23 μm, 24 μm, and/or 25 μm. In some embodiments, the cells attached to a primary attachment surface may have a ratio of any two dimension (length, width, or height and herein aspect ratio) of about 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100.

Cell separation features may include structural features including pockets, such as depressions, indents, partial voids, or similar features in or on a cell system surface or substrate. Pockets may include microwells. As used herein, the term “microwell” is used to refer to pockets significantly smaller (e.g., lower volume) than those associated with typical assay wells and may include pockets within larger wells. Microwells may include grooves in cell culture surfaces. Such grooves may be formed by one or more of etching, micro molding, milling, and 3D printing. Pockets may include a variety of shapes and depths. Pockets may be cylindrical in shape. Some pockets may be linear, rectangular, rounded, oblong, or irregular in shape. Pocket side walls may be straight, curved, irregular, or tapered. A tapered wall may be tapered such that the top of the pocket has a larger diameter than the bottom of the pocket, or vice versa. The shape and size of the pockets may assist in maintaining cell attachment. For example, as a cell grows in a tapered pocket having a smaller top diameter than bottom diameter, the cell may be blocked from floating up to the top of the pocket if the cell's diameter exceeds that of the pocket top diameter. In some embodiments, the cells may be blocked from floating if they undergo hypertrophy. Microwells may be sized and shaped to allow at least one cell to reside in the pocket and to prevent accumulation of multiple cells.

Microwells may have a diameter from about 5 μm to about 500 μm, e.g., from about 10 μm to about 300 μm, from about 15 μm to about 200 μm, from about 20 μm to about 100 μm, and from about 25 μm to about 75 μm. Microwells may have a height from about 1 μm to about 100 μm, for e.g., from about 2 μm to about 20 μm, and from about 4 μm to about 10 μm.

Cell separation features may include structural features such as projections on a cell system surface or substrate, including cell culture surfaces. Individual cells may be positioned on a top face of each projection and/or between projections. Projections may be formed from one or more of micro molding, 3D printing, etching and milling. Projections may have a variety of dimensions and shapes. Projections may include pillars. The cross-section of the projections may be round, polygonal or irregular in shape. Projections may be evenly dimensioned around an outer wall perimeter or may be tapered or irregularly dimensioned. Projections may have flat or recessed top surfaces.

Projections may have a width from about 5 μm to about 500 μm, e.g., from about 10 μm to about 300 μm, from about 15 μm to about 200 μm, from about 20 μm to about 100 μm, and from about 25 μm to about 75 μm. Projections may have a height from about 1 μm to about 100 μm, from about 2 μm to about 20 μm, and from about 4 μm to about 10 μm.

In some embodiments, cell separation features comprise one or more coating layers to facilitate cell positioning in cell systems. Cell system coating layers may be applied at specific locations and/or in specific patterns in a cell culture system to promote and facilitate cell positioning and attachment. In some embodiments, the cell system coating layers may be applied to limit cell size (e.g. preadipocyte and/or adipocyte cell size) within the cell systems.

Coating layers may be used in conjunction with other cell separation features, including structures such as pockets and/or projections. Coating layers may be applied in, on and/or around cell separation structures to desired cell attachment areas. Cell attachment areas may include top surfaces of projections and/or spaces between adjacent projections. Cell attachment areas may include some or all of the surface area in pockets, including the bottom surface and/or the side walls.

In some embodiments, coating layers may be used to support seeding, growth, differentiation, maturation, and/or progression to hypertrophy of preadipocytes (e.g., adipose stem cells and mesenchymal stem cells) and adipocytes. Such coating layers may include extracellular matrix proteins. Extracellular matrix proteins useful as coating proteins may include, but are not limited to, collagen, laminin, fibronectin, gelatin, or fragments or combinations thereof.

Patterned coating layers of extracellular matrix proteins may be used to geometrically constrain preadipocytes such that they may have a rounded shape (relative to cells that can spread unimpeded on a cell culture substrate).

Constraining cell size destabilizes the cytoskeleton and predisposes preadipocytes to become adipocytes. Conversely, when preadipocytes are unconstrained and allowed to spread, adipocyte differentiation may be inhibited in favor of an osteocyte fate. While starting with high density cultures of preadipocytes may be efficient for differentiating adipocytes from stem cells, it dramatically slows their size growth (i.e., hypertrophy) and maturation. Applying coating layers in microscopic patterns on cell culture surfaces (also termed “micropatterning”) allows adipocytes to grow in size much more quickly, while keeping cells rounded to maximize differentiation efficiency. Micropatterning individual preadipocytes allows differentiation of the preadipocytes to become independent of cell density while prolonging culture longevity and supporting hypertrophy. Importantly, adjusting cell spacing in the pattern allows control of hypertrophy independent of time in culture, media composition, or genetic manipulation. In some embodiments, coating layer micropatterning may be carried out by microcontact printing, e.g., as described in United States Publication Numbers US 2020/0248116 and US 2018/327770, the contents of each of which are herein incorporated by reference in their entirety. In some embodiments, cell systems may include cell culture surfaces that are commercially available with pre-microprinted coating layers. In some embodiments, substrates with microprinted coating layers may be prepared by photopatterning. As used herein, the term “photopatterning” refers to a form of coating layer application that involves application of a layer of photolabile coating material that may be subjected to laser light to remove coating from certain locations, e.g., to yield a micropattern. Adipocytes naturally transition from fibronectin attachment to laminin during differentiation. Coating layers may be constructed accordingly to promote differentiation. In some embodiments, coating layers may include decellularized or synthetic extracellular matrix preparations and components to promote cell attachment.

In some embodiments, coating layers may be applied to the area surrounding substrates or a cell culture surface. Such coating layers may be utilized to deter cell positioning outside the substrate or cell culture surface. In some embodiments, coating layers may be hydrophobic to prevent cell attachment outside the substrate or cell culture surface.

In some embodiments, coating layer components (e.g., proteins) may be applied to substrates at a specific amount per area being coated. In some embodiments, coating layer components are applied in an amount of from about 1 ng to about 500 μg per square centimeter of substrate (e.g., in an amount of from about 1 ng/cm² to about 20 ng/cm², from about 5 ng/cm² to about 50 ng/cm², from about 10 ng/cm² to about 100 ng/cm², from about 20 ng/cm² to about 200 ng/cm², from about 150 ng/cm² to about 500 ng/cm², from about 250 ng/cm² to about 750 ng/cm², from about 0.5 μg/cm² to about 10 μg/cm², from about 5 μg/cm² to about 50 μg/cm², from about 10 μg/cm² to about 100 μg/cm², from about 20 μg/cm² to about 200 μg/cm², and from about 150 μg/cm² to about 500 μg/cm².

In some embodiments, coating layers are applied to promote arrangement of individual cell system cells to include spacing between adjacent cells of from about 5 μm to about 500 μm (e.g., from about 5 μm to about 40 μm, from about 10 μm to about 50 μm, from about 20 μm to about 60 μm, from about 30 μm to about 70 μm, from about 40 μm to about 80 μm, from about 50 μm to about 90 μm, from about 45 μm to about 100 μm, from about 50 μm to about 110 μm, from about 55 μm to about 120 μm, from about 60 μm to about 130 μm, from about 65 μm to about 140 μm, from about 70 μm to about 150 μm, from about 75 μm to about 160 μm, from about 80 μm to about 170 μm, from about 85 μm to about 180 μm, from about 90 μm to about 190 μm, from about 95 μm to about 200 μm, from about 100 μm to about 210 μm, from about 115 μm to about 220 μm, from about 125 μm to about 240 μm, from about 135 μm to about 260 μm, from about 145 μm to about 280 μm, from about 155 μm to about 300 μm, from about 165 μm to about 320 μm, from about 175 μm to about 340 μm, from about 185 μm to about 360 μm, from about 195 μm to about 380 μm, from about 205 μm to about 400 μm, from about 215 μm to about 420 μm, from about 225 μm to about 440 μm, from about 235 μm to about 460 μm, or from about 245 μm to about 500 μm). In some embodiments, the spacing between adjacent cells may be essentially equal. In other embodiments, the spacing between adjacent cells may be variable.

Cell systems may be configured for high throughput simultaneous analysis of multiple factors. Such systems may incorporate multiple wells with individualized cell system components that may be incorporated individually or as a single unit (e.g., a layer).

### Cell Support Layers

Cell systems of the present disclosure may include cell support layers. As used herein, the term “cell support layer” refers to a structure used to maintain cell position in a cell system. Cell support layers may maintain cell position by preventing cells from detaching from a cell culture surface, for example if cells become buoyant. Cell support layers may allow cells to form new attachment points, for example on the cell support layers. Cell support layers may provide specific microenvironmental cues.

In some embodiments, cell support layers comprise cell support members for placement in and/or on cell separation systems for maintaining cell position in cell separation systems. Cell support members may comprise a porous material. When the cell support member is placed over a cell in a cell separation system, the porous material, which has pores smaller in size than the average cell size, prevents the cell from moving through the porous material, while still allowing fluid communication through the porous material to allow the introduction of components to the cell, including but not limited to cell culture media, cell system factors, test factors and therapeutic agents.

The porous material may have a porosity of less than 100%, for example, less than 95%, less than 90%, less than 80%, less than 70%, less than 60%, less than 50%, less than 40%, less than 30%, less than 20%, less than 10%, less than 5%. As a non-limiting example, the porosity may be from about 20% to about 50%. The average size of the pores, measured at their largest cross-section, less than 10 μm, for example 1 μm, 2 μm, 3 μm, 4 μm, 5 μm, 6 μm, 7 μm, 8 μm, or 9 μm. The average pore size may be from about 1 μm to about 5 μm.

The porous material may be a membrane, such as a nanofiber membrane, a track etched plastic membrane or a cellulose acetate membrane. The nanofiber membrane may be a mesh comprising overlapping fibers with spaces between the fibers forming the pores. As used herein, the term “nanofiber” refers to a fiber of material with a diameter of from about 10 nm to about 5000 nm. The nanofiber may include a diameter of from about 200 nm to about 1000 nm. The nanofiber material may be formed from any one of or combination of controlled spinning of nanofibers, lithography, deposition and crystal growth.

In some embodiments, cell support members are sized and shaped to fit into or over wells in a substrate, the wells containing cell separation features and an arrangement of cells. The outer perimeter of the cell support member may have a shape and dimensions to fit snugly into or over the well to contact the sidewalls of the well and to be positioned over the cells arranged in the bottom of the well. When the cell support member is inserted into the well, the porous material may be positioned at or near the bottom of the well above the cells in the pocket. The porous material may contact the cells or be spaced apart from the cells. Porous material may be prepared by assembling polymer fibers over the cell separation features e.g. pocket or an array.

Cell support members may be substantially planar. The outer perimeter of cell support members may have substantially the same shape as the inner walls of wells containing cell separation features and an array of individual cells, but with a slightly smaller diameter, which allows the cell support members to be inserted into the wells. The cell support members may be circular, polygonal or irregular in shape.

Cell support members may comprise a body with one or more holes, wherein the porous material covers the hole(s). The body may be a rigid material that supports a flexible porous material, and the one or more holes allows for fluid communication through the cell support member in the case where the body material is not porous. There may be one hole or multiple holes. Alternatively, the porous material may be rigid such that a body is not needed for support, in which case the cell support member may be composed of the porous material. In one embodiment, the porous material may be a disc. In some embodiments, the porous material can be a material containing a network of inter-connected cavities.

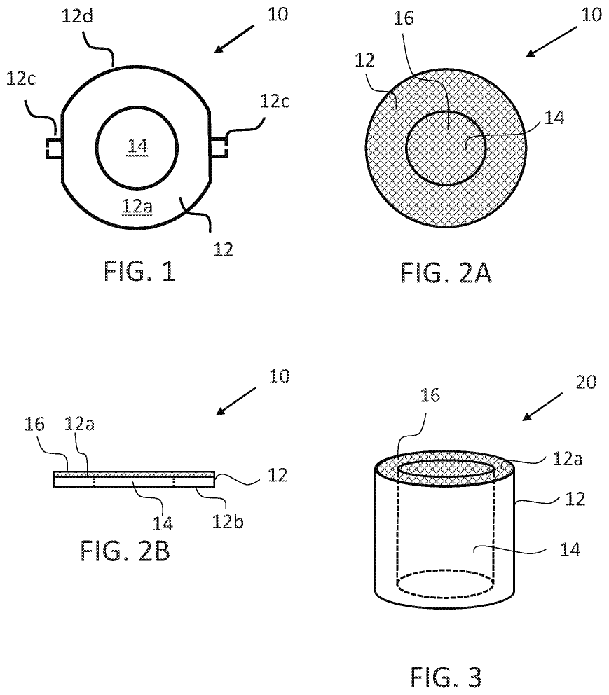

Examples of planar support members 10 are shown in FIGS. 1, 2A, 2B, wherein the bodies are substantially circular (i.e., ring-shaped). The support member comprises a support member body 12 having a substantially flat upper face 12a and a substantially flat lower face 12b, with a central hole 14 extending between the upper face and the lower face. There may be one or more tabs 12c protruding from an outer edge 12d of the support member body for engaging with a cell substrate, for example with one or more walls of a well containing cell separation features and an array of cells. The engagement of the tabs may prevent movement of the support member with respect to the well after placement of the support member. There may be any number of tabs, including one tab, two tabs, three tabs, four tabs, or more. The tabs may be evenly spaced around the outer edge of the support member body. Alternatively, the support member body may not include tabs (such as in FIG. 2A), and the outer edge of the support member body may uniformly contact the well for engagement with the well.

The porous material covering the hole may also cover at least part of one face of the support member body. In one embodiment, the hole may cover a whole face of the support member body. The porous material is 16 is shown covering the upper face 12a in FIGS. 2A, 2B.

The diameter of cell support member bodies may be from about 0.1 μm to about 1 cm. In some embodiments, the diameter of cell support member bodies may be less than 10 μm, for example 1 μm, 2 μm, 3 μm, 4 μm, 5 μm, 6 μm, 7 μm, 8 μm, or 9 μm. In some embodiments, the diameter of the cell support member bodies may be from about 1 μm to about 5 μm. The diameter of cell support member bodies may be from about 1 mm to about 10 mm, for e.g., 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm. In some embodiments, the diameter of the cell support member bodies may be from about 5.5 mm to about 6.5 mm.

The diameter of the one or more holes may be from about 0.1 μm to about 10 mm. In some embodiments, the diameter of the hole may be less than 10 μm, for example 1 μm, 2 μm, 3 μm, 4 μm, 5 μm, 6 μm, 7 μm, 8 μm, or 9 μm. In some embodiments, the diameter of the hole may be from about 1 μm to about 5 μm. The diameter of the hole may be from about 1 mm to about 10 mm, for e.g., 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm. In some embodiments, the diameter of the hole may be from about 5.5 mm to about 6.5 mm.

The support member bodies may comprise a thermoplastic material.

An example of a tubular support member 20 is shown in FIG. 3, wherein the hole 14 extends as a channel through the support member body 12 between a first end and a second end, with one end of the hole covered in the porous material 16, which in this example covers the top face 12a of the support member body. The porous material may cover at least one face, or at least part of one face, of the tubular body. The tube may be cylindrical or non-cylindrical in shape. The tube may be sized and shaped to fit with a cell substrate, for example to be inserted into a well containing cell separation features and an array of individual cells.

In some embodiments, the tubular support may include a single central hole. In some aspects, the porous material may cover one or more holes.

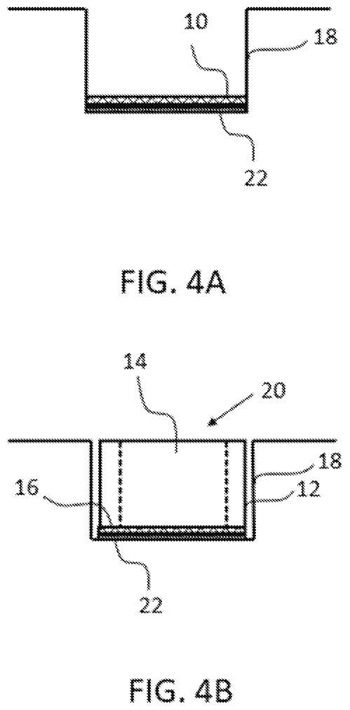

FIGS. 4A, 4B illustrate examples of how a cell support member may be aligned and placed with respect to a well containing cell separation features and an arrangement of individual cells. FIG. 4A illustrates a substantially planar support member 10 inserted into a well 18 containing an arrangement of cells 22 separated by cell separation features at the bottom of the well. FIG. 4B illustrates a tubular support member 20 having a hole 14 extending through the support member body 12, with a porous material 16 covering one end of the hole 14 and the bottom face of the support member. The tubular support member is positioned in a well 18 containing an arrangement of cells 22 separated by cell separation features at the bottom of the well 18. There is a gap 24 illustrated between the well walls and the tubular support member body 12, however in some embodiments there is no gap and the tubular support member body is in contact with the microwell walls.

In some embodiments, the cell support layer comprises an array of cell support members. The array of cell support members may comprise an array body retaining a plurality of support members. The support members may be retained at a position and spacing to facilitate placement of the support members over a cell separation system. The array of cell support members may allow for simultaneous manipulation and positioning of multiple support members. The array of cell support members can be positioned with respect to a cell separation system such that each support member in the array is aligned over an array of cell separation feature, including an array of pockets or pillars.

The array body may comprise a sheet of material, such as a thermoplastic material. The support members in the array may be planar support members or tubular support members. For planar support members, the top and bottom faces of the support members may be substantially even with the top and bottom surfaces of the array body. For tubular support members, the tubular member bodies may extend from the top surface or the bottom surface of the array body and may be prepared by molding or 3D printing followed by coating or filling with the porous material. The array body may support the array of support members on the cell separation system to allow the support members to be positioned over cell separation features.

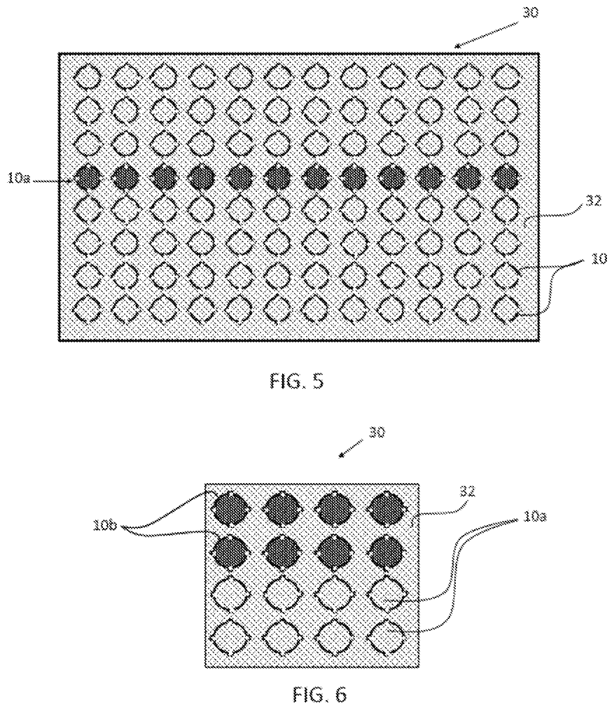

An example of an array 30 of planar support members is shown in FIG. 5. The array comprises a continuous sheet of material forming the array body 32 that retains a plurality of planar support members 10 that are ring-shaped. In FIG. 5, one row of cell support members 10a have been disengaged from the array body. FIG. 6 illustrates another example showing a portion of an array 30 wherein some planar support members 10a have been inserted into wells containing an arrangement of cells and cell separation features below the array, while some cell support members 10b are still retained in the array body 32.

For an array of tubular cell support members, the array may be positioned with respect to a cell separation system such that the tubular support members extend into wells containing an arrangement of cells and cell separation features. For example, when a lower face of the array body is supported on an upper face on the cell separation system, tubular support members may extend into wells and cover projections of the cell separation system. The length of the tubular support members may allow the distal ends (i.e. the ends furthest from the array body) of the support members to be at or near the bottom surface of the well, above the cells.

Using an array of cell support members allows for high throughput configurations of cell systems, wherein a plurality of cell support members can be aligned and engaged with a plurality of cell separation structures quickly, either serially or simultaneously.

Cell support layers may be composed of synthetic and/or biogenic (meaning produced or otherwise deriving from living organisms) components. In some embodiments, cell support layers include synthetic and/or biogenic polymers. Synthetic polymers may include, but are not limited to, one or more of a poly(urethane), a poly(siloxane), a poly(ethylene), a poly(vinyl pyrrolidone), a poly(2-hydroxy ethyl methacrylate), a poly(N-vinyl pyrrolidone), a poly(methyl methacrylate), a poly(vinyl alcohol), a poly(acrylic acid), a polyacrylamide, a poly(ethylene-co-vinyl acetate), a poly(ethylene glycol), a poly(methacrylic acid), a polylactides (PLA), a polyglycolide (PGA), a poly(lactide-co-glycolide) (PLGA), a polyanhydride, a polyphosphazene, a polygermane, a polyorthoester, a polyester, a polyamide, a polyolefin, a polycarbonate, a polyaramide, a polyimide, a polycaprolactone (PCL), and a copolymer, derivative, or combination thereof. Biogenic polymers may include, but are not limited to, one or more of a protein, a polypeptide, a polysaccharide, a lipid, a nucleic acid, and a glycosaminoglycan. Biogenic polymers may include, but are not limited to, one or more of silk, fibroin, sericin, keratin, alpha-keratin, beta-keratin, alginate, elastin, fibrillin, fibrillin-1, fibrillin-2, fibrillin-3, fibrillin-4, fibrinogen, fibrin, fibronectin, laminin, collagen, collagen I, collagen II, collagen III, collagen IV, collagen V, collagen VI, vimentin, neurofilament, light chain neurofilament (NF-L), medium chain neurofilament (NF-M), heavy chain neurofilament (NF-H), amyloid, alpha-amyloid, beta-amyloid, actin, myosin, titin, gelatin, chitin, hyaluronic acid, D-glucuronic acid, and D-N-acetylglucosamine.

Cell support layers may include polymer fibers. Polymer fibers may include a diameter of from about 10 nm to about 5000 nm (e.g., from about 10 nm to about 25 nm, from about 20 nm to about 50 nm, from about 30 nm to about 75 nm, from about 40 nm to about 80 nm, from about 50 nm to about 100 nm, from about 60 nm to about 150 nm, from about 70 nm to about 160 nm, from about 80 nm to about 170 nm, from about 90 nm to about 180 nm, from about 100 nm to about 190 nm, from about 110 nm to about 200 nm, from about 120 nm to about 210 nm, from about 130 nm to about 220 nm, from about 140 nm to about 240 nm, from about 150 nm to about 260 nm, from about 160 nm to about 280 nm, from about 170 nm to about 300 nm, from about 180 nm to about 320 nm, from about 190 nm to about 340 nm, from about 200 nm to about 360 nm, from about 210 nm to about 380 nm, from about 220 nm to about 400 nm, from about 230 nm to about 420 nm, from about 240 nm to about 440 nm, from about 250 nm to about 460 nm, from about 260 nm to about 500 nm, from about 270 nm to about 600 nm, from about 280 nm to about 700 nm, from about 290 nm to about 800 nm, from about 300 nm to about 900 nm, from about 500 nm to about 1000 nm, from about 750 nm to about 2000 nm, from about 1250 nm to about 3000 nm, from about 2500 nm to about 4000 nm, or from about 3500 nm to about 5000 nm). In some embodiments, polymer fibers may include a diameter of from about 200 nm to about 1000 nm.

Cell support layers may include at least about 50 wt % synthetic and/or biogenic polymer fibers (e.g., from about 50 wt % to about 60 wt %, from about 55 wt % to about 65 wt %, from about 60 wt % to about 70 wt/o, from about 65 wt % to about 75 wt %, from about 70 wt % to about 80 wt %, from about 75 wt % to about 85 wt %, from about 80 wt % to about 90 wt %, from about 85 wt % to about 95 wt %, or from about 90 wt % to about 100 wt % synthetic and/or biogenic polymer fibers). In some embodiments, cell support layers may include from about 1 to about 49 wt % synthetic and/or biogenic polymer fibers (e.g., from about 1 wt % to about 5 wt %, from about 2 wt % to about 6 wt %, from about 3 wt % to about 7 wt %, from about 4 wt % to about 8 wt %, from about 5 wt % to about 9 wt %, from about 6 wt % to about 10 wt %, from about 7 wt % to about 11 wt %, from about 8 wt % to about 12 wt %, from about 9 wt % to about 13 wt %, from about 10 wt % to about 14 wt %, from about 12 wt % to about 15 wt %, from about 14 wt % to about 17 wt %, from about 16 wt % to about 19 wt %, from about 18 wt % to about 21 wt %, from about 20 wt % to about 23 wt %, from about 22 wt % to about 25 wt %, from about 24 wt % to about 27 wt %, from about 26 wt % to about 29 wt %, from about 28 wt % to about 31 wt %, from about 30 wt % to about 33 wt %, from about 32 wt % to about 35 wt %, from about 34 wt % to about 37 wt %, from about 36 wt % to about 39 wt %, from about 38 wt % to about 41 wt %, from about 40 wt % to about 43 wt %, from about 42 wt % to about 45 wt %, from about 44 wt % to about 47 wt %, or from about 46 wt % to about 49 wt % synthetic and/or biogenic polymer fibers).

Polymer fibers may include PCL. Such polymer fibers may include at least about 50 wt % PCL (e.g., from about 50 wt % to about 60 wt %, from about 55 wt % to about 65 wt %, from about 60 wt % to about 70 wt %, from about 65 wt % to about 75 wt %, from about 70 wt % to about 80 wt %, from about 75 wt % to about 85 wt %, from about 80 wt % to about 90 wt %, from about 85 wt % to about 95 wt %, or from about 90 wt % to about 100 wt % PCL).

Some polymer fiber may include gelatin. Such polymer fibers may include from about 1 to about 49 wt % gelatin (e.g., from about 1 wt % to about 5 wt %, from about 2 wt % to about 6 wt %, from about 3 wt % to about 7 wt %, from about 4 wt % to about 8 wt %, from about 5 wt % to about 9 wt %, from about 6 wt % to about 10 wt %, from about 7 wt % to about 11 wt %, from about 8 wt % to about 12 wt %, from about 9 wt % to about 13 wt %, from about 10 wt % to about 14 wt %, from about 12 wt % to about 15 wt %, from about 14 wt % to about 17 wt %, from about 16 wt % to about 19 wt %, from about 18 wt % to about 21 wt %, from about 20 wt % to about 23 wt %, from about 22 wt % to about 25 wt %, from about 24 wt % to about 27 wt %, from about 26 wt % to about 29 wt %, from about 28 wt % to about 31 wt %, from about 30 wt % to about 33 wt %, from about 32 wt %/o to about 35 wt %, from about 34 wt % to about 37 wt %, from about 36 wt % to about 39 wt %, from about 38 wt % to about 41 wt %, from about 40 wt % to about 43 wt %, from about 42 wt % to about 45 wt %, from about 44 wt % to about 47 wt %, or from about 46 wt % to about 49 wt % gelatin). In some embodiments, polymer fibers include about 70 wt % PCL and about 30 wt % gelatin.

In some embodiments, cell support layers may include polymer fiber sheets. In some instances, polymer fiber sheet cell support layers may include a frame. In some embodiments, the thickness of the frame may be from about 0.01 mm to about 10 mm, for e.g. 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 5 mm, and/or 10 mm. Such cell support layers may be particularly well suited for use with cell systems that include cell culture wells. In some embodiments, the polymer fiber sheet cell support layer may be attached to a frame having an outer diameter similar to that of a cell culture well. The polymer fiber sheets may be collected on a surface of a frame; or the polymeric fibers may be collected on a surface of multiple frames connected to or attached to each other to and the collection of the polymer fiber sheets forms a large fiber layer. Framed polymer fiber sheets may be prepared with frames that facilitate reversible seating in cell culture wells (i.e., such frames may be configured for insertion into wells with a light force that may result in a tight fit, but loosely enough to facilitate manual removal). Frames may be made of flexible materials to facilitate reversible seating (e.g., thermoplastic). In some embodiments, framed polymer fiber sheets include those described in United States Publication Number US 2020/0248116, the contents of which are incorporated herein by reference in their entirety, e.g., pages 7 and 27, and FIGS. 2A-2E of the corresponding application as originally filed). Reversible seating in cell culture well may be useful for analyzing cells in vitro.

### Cell System Factors

In some embodiments, cell systems of the present disclosure include cell system factors. As used herein, the term “cell system factor” refers to any substance or compound included in or introduced to a cell system that affects cell system cells (e.g., supports, stabilizes, or modulates one or more cell activities, e.g., survival, growth, proliferation, differentiation, etc.). Cell system factors may include, but are not limited to, one or more of a molecule, a compound, a polypeptide, a nucleic acid, a lipid, a carbohydrate, a liquid, a gas, a particle, and a cell. Cell system factors may include cell modulators. As used herein, the term “cell modulator” refers to any compound or substance that alters one or more cell activities, e.g., growth, proliferation, differentiation, etc. Cell modulators may include cell signaling molecules. Cell signaling molecule may include, but are not limited to, growth factors, cytokines, hormones, steroids, nutrients, and receptor ligands. In some embodiments, cell system factors may include, but are not limited to, insulin, dexamethasone, and rosiglitazone.

Cell system factors may include particles that include one or more molecules, compounds, polymers, polypeptides, nucleic acids, lipids, and/or carbohydrates. Particles may include polymer particles, i.e., particles that include polymers (e.g., any of the polymer types described herein). Polymer particles may include spherical or non-spherical shapes. In some embodiments, polymer particles include Janus spheres.

In some embodiments, cell system factors may include any of those described previously (e.g., see United States Publication Numbers US 2020/0248116 and US 2018/327770, U.S. Pat. No. 9,410,267, or International Publication Number WO2018148669, the contents of each of which are herein incorporated by reference in their entireties).

### Layered Devices

Cell systems may include devices comprising layers that may be assembled to form cell systems. In some embodiments, the present disclosure provides devices for assembly of cell systems according to any of those described herein. Such devices may include layers that may be assembled to form cell systems. In some embodiments, at least one of the layers of the devices described herein may include an arrangement of cells (herein referred to as a cell containing layer). Devices may include layers individually or may combine one or more of substrates, cell separation features, cell culture surfaces, and cell support layers. Devices may be configured for high throughput analysis by incorporating multiple wells into the layered design. Cell support layers utilized according to such high throughput configurations may be formatted as a sheet that can be applied simultaneously to wells. The sheets may facilitate cell support layer application to cell system wells via pressing or punching or the sheets may be molded to fit cell support layers directly into wells to cover and support cell system wells. In some embodiments, the order and combination of layers within the device may be customized depending on the particular method of use.

Devices may include one or more reservoir layers that can be positioned above cell containing layers and/or cell support layers and that may facilitate application of cell culture media, cell system factors, test factors, therapeutic agents, or other components being introduced to cell systems.

Alignment components may be incorporated into one or more device layers to achieve lateral alignment of the layers. The alignment component may include structures on one or more layers. The alignments structures may include one or more projections (e.g. dowels, tabs, shoulders, edges) on the first device layer and corresponding recesses (e.g., holes, slots, grooves) on the second device layer that can be fit together to align the device layers. For example, a base layer with projections (e.g. dowel) upwards from the top face may be incorporated. Cell culture surfaces, cell support layers, and/or reservoir layers, may include recesses (e.g. holes) positioned to receive the dowels from the base layer that can be fit together to align the different device layers. As a non-limiting example, the layered device may include a base cell layer that may include one or more cell separation structures with a pocket e.g. microwell. The microwell may include a cylindrical recess on the top surface configured for containing individual cells. As a non-limiting example, a recess (e.g., a trough) may surround the cell system to capture cell culture medium leakage or spills. As a non-limiting example, the layered devices may contain a reservoir layer with holes on the top face and the bottom face that may be connected by channels. By placing such reservoir layers on a flat surface, wells may be created within the reservoir layer to contain cell culture medium.

The layered devices may be prepared for preadipocyte differentiation and/or adipocyte culture. Pre-adipocytes require differentiation in a culture environment where they are separated into individual cells adhered a defined distance apart. A cell system designed for pre-adipocyte differentiation may include cell separation system features that contain an array of circular recesses that each include a patterned coating layer to define attachment locations for individual cells at specific locations. Mature adipocytes undergoing hypertrophy become buoyant and lose their adhesion over long term culture. A cell support layer may be included in the layered devices described herein to maintain adipocyte cell position as well as provide microenvironmental cues to the adipocytes. Layered devices may also include channel features, to allow for microfluidic flow in between the layers and its components within.

Layers of devices described herein may be combined and replaced as needed. In some embodiments, the cell systems may include layered devices containing a cell system substrate with one or more cell separation features (e.g. a coating layer) and a reservoir layer filled with a first cell culture medium. As a non-limiting example such systems may be used to differentiate progenitor cells (e.g. a preadipocyte) to mature cell types (e.g. adipocyte) and the first cell culture medium may promote the differentiation. Following differentiation, the first cell culture medium may be replaced with a second cell culture medium. As a non-limiting example, the second cell culture medium may be used to maintain the culture of mature cells (e.g. adipocytes). A cell support layer may also be placed between the cell system substrate with one or more cell features, and the reservoir layer to maintain cell position in the cell system e.g. if cells become buoyant.

Different layers may also include one or more cell system factors. One or more cell system factors containing molecules, compounds, polypeptides, nucleic acids, lipids and/or carbohydrates may be prepared as particles and placed between any of the layers of the devices described herein.

Layered devices may include one or more physical, chemical, biological, optical, thermal, magnetic, and/or electronic components, whose selection and identity is optimized to the intended method of use. For example, the devices may include an array LEDs and associated microcontrollers or electronics.

In some embodiments, devices may include more than one layer containing cells or tissues. The same cell type may be present in the more than one layers. In some embodiments, each cell containing layer may include a different type. Devices may also include cells containing a combination of cell types or tissues. The layers containing cells may be positioned anywhere within the layered device. As a non-limiting example, the devices may include two cell containing layers. The first cell containing layer may include preadipocyte or adipocytes, whereas the second cell containing may include islet cells. The second cell containing layer may be introduced between the first cell containing layer and the reservoir layer. Each cell containing layer may alternatively include a companion reservoir layer where the cell containing layer may be configured to have access to the cell culture medium in the reservoir layer.

Material for each layer in the device may be the same for two or more layers or may be different. In one embodiment, the layers may be prepared from materials such as, but not limited to plastics (e.g. acrylic), metal, glass, or elastomers (e.g. PDMS). Layers may be formed by one or more of laser cutting, milling, embossing, molding, 3D printing, lithography, etching, and/or deposition.

Devices may include gaskets between reservoir layers and cell support layers to avoid leakage of reservoir elements (e.g., into neighboring cell system wells). Devices may include gaskets between cell support layers and base layers of devices. In some embodiments, devices may include one or more clamping layers to secure two or more device layers together. In some embodiments, clamping force required to allow gaskets to experience 10-20% deformation in the direction of clamping force application, may be applied.

The disclosure also covers methods for combining different layers within the devices described herein. In one embodiment, layers may be manually assembled and clamped together using custom C-shaped clamps with fixed distance between their arms. Multiple C-shaped clamps may be required for different layer configurations. In some embodiments, each layer may include one or more threaded holes. The layers of the device may be screwed together after manual assembly. The base layer may include threaded holes to and/or structures to bring the layers together. In another embodiment, one or more layers of a layered devices may include recess (e.g., grooves) along the length of their sides intended to fit over corresponding projections within a holding shelf. Cell systems may include one or more shelves. Multiple shelves may be positioned along a vertical frame with rails, such that the shelves can move up and down when layers are mounted in them. In some embodiments, the shelves may be locked in position within the vertical frame. Movement of shelves along the rails of the vertical frame may be manual, or may be actuated in some passive or active fashion. For instance, the shelves may be loaded with springs, such that the layers may be pushed together and locked in place. In some aspects, the top shelf may be spring loaded such that it may be drawn downwards, applying force to any layers and or/shelves underneath. In another embodiment, the shelves may be actuated through the use of motors, such that each shelf may be moved independently of any of the other shelves. Dedicated gasket shelves may also be employed such that a gasket layer is mounted on a rigid (e.g. metal) plate.

### II. Methods

### Cell System Preparation

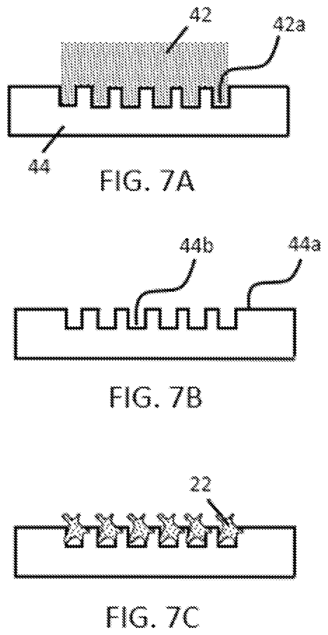

In some embodiments, the present disclosure provides methods of preparing cell systems. Such methods may include preparing cell systems that include cell separation systems. The cell systems may be prepared by forming cell separation features in a substrate; applying a coating layer to areas of the substrate; associating a cell with the coating layer; and/or optionally covering the cell with a cell support layer. The cell separation features may be projections and/or pockets, including microwells. Cell separation features may be formed by one or more of etching, micromolding, 3D printing and/or milling substrates. Substrates may include cell culture surfaces. Cell culture surfaces may include one or more of cell culture dishes, cell culture wells, polymers, including plastics and elastomers, resins, metals and glass. In some embodiments, the cell culture surfaces may include an elastomer. Forming cell separation features may be done by treating a substrate with a solvent to dissolve a top surface of the substrate and forming the top surface to have the desired cell separation features. The forming may be done by imprinting the top surface using a stamp having a structure shaped in the negative of the desired cell separation features. The solvent may be applied to the top surface of the substrate and/or the stamp surface prior to imprinting. One example of this is shown in FIGS. 7A, 7B, 7C showing a method for forming cell separation structures using a stamp. First, the stamp 42 comprising projections 42a and substrate 44 are pressed together to form cell separation structures comprising pockets 44b in the top surface 44a of the substrate, shown in FIG. 7B. After forming the pockets 44, cells 22 can be associated with each pocket. Alternatively, the stamp may contain recesses for forming projections in the substrate for supporting cells on top of the projections and/or between projections

In some embodiments, forming cell separation features may be performed by pouring a dissolved material into a mold shaped as the negative of the desired cell separation features; and allowing the solvent to evaporate to leave a solid substrate containing the desired cell separation features.

Coating layers may include coating proteins. Coating proteins may include extracellular matrix proteins. Extracellular matrix proteins may include collagen, laminin, fibronectin, gelatin, or a fragment of combination thereof. Applying coating layers may include one or more of microcontact printing, photolabile polymer printing, and/or stamping. Coating layers may be applied to cell separation systems by positioning blocking masks over substrates to align blocking mask holes or gaps with the desired areas to apply the coating layer to and then applying coating layers to the blocking mask. According to such methods, the desired areas for coating remain coated while the remainder of the substrate may be free of the coating when the blocking mask may be removed. The desired areas for coating may include the bottom and/or walls of pockets, on top of projections, and/or in areas surrounding projections.

In some embodiments, from about 0.01 μg to about 50 μg of coating protein (or other coating layer component) may be applied per square centimeter of surface area being coated (e.g., pocket surface area or top face of projection). In some embodiments, from about 0.01 μg/cm² to about 10 μg/cm², from about 0.05 μg/cm² to about 20 μg/cm², from about 0.1 μg/cm² to about 30 μg/cm², from about 1 μg/cm² to about 40 μg/cm², or from about 10 μg/cm² to about 50 μg/cm² of coating protein (or other coating layer component) may be—applied.

Associating cells with coating layers may include contacting coating layers with solutions of cells, allowing the cells to associate with coating layers, and removing unassociated cells. Cell solutions used for association may include cell concentrations suitable to provide from about 1 to about 10,000 cells per square millimeter of pocket surface area. In some embodiments, cell solutions include concentrations suitable to provide from about 1 cell/mm² to about 20 cells/mm², from about 5 cells/mm² to about 50 cells/mm², from about 10 cells/mm² to about 100 cells/mm², from about 20 cells/mm² to about 200 cells/mm², from about 100 cells/mm² to about 500 cells/mm², from about 150 cells/mm² to about 1000 cells/mm², from about 200 cells/mm² to about 1500 cells/mm², from about 250 cells/mm² to about 2000 cells/mm², from about 300 cells/mm² to about 2500 cells/mm², from about 350 cells/mm² to about 3000 cells/mm², from about 400 cells/mm² to about 3500 cells/mm², from about 450 cells/mm² to about 4000 cells/mm², from about 500 cells/mm² to about 4500 cells/mm², from about 550 cells/mm² to about 5000 cells/mm², from about 600 cells/mm² to about 5500 cells/mm², from about 650 cells/mm² to about 6000 cells/mm², from about 700 cells/mm² to about 6500 cells/mm², from about 750 cells/mm² to about 7000 cells/mm², from about 800 cells/mm² to about 7500 cells/mm², from about 850 cells/mm² to about 8000 cells/mm², from about 900 cells/mm² to about 8500 cells/mm², from about 950 cells/mm² to about 9000 cells/mm², from about 1000 cells/mm² to about 9500 cells/mm², or from about 2000 cells/mm² to about 10000 cells/mm². In some embodiments, the cells may include one or more of stem cells, progenitor cells, mesenchymal progenitor cells, preadipocytes, and mature adipocytes. In some embodiments, coating layers may be applied to the area surrounding substrates or a cell culture surface. Such coating layers may be utilized to deter cell positioning outside the substrate or cell culture surface. In some embodiments, coating layers may be hydrophobic to prevent cell attachment outside the substrate or cell culture surface.

### Cell Support Layers Preparation

Cell systems may be prepared to include cell support layers. Cell support layers may be applied after association of cells with cell systems.

In some embodiments, the present disclosure provides methods of preparing cell support layers. Such methods include preparing cell support layers that include cell support members for maintaining and/or affecting cell position, including maintaining cell attachment to cell culture surfaces and/or promoting new cell attachment points. Such methods include preparing individual cell support members and preparing arrays of cell support members. Cell support members may be planar or tubular.

In some embodiments, cell support members are prepared by providing a sheet of material; cutting cell support member bodies having desired shapes and dimensions in the sheet of material, including forming one or more holes in the support member bodies; and covering the holes with a porous material. The cell support members may be prepared as an array of support members, which may be planar support members.

Cutting the support member bodies may include cutting around the perimeters of the support member bodies. When cutting around the perimeters of the support member bodies, a section of the perimeter may be left uncut such that the support member bodies remain attached to the sheet of material, which forms the array body. The uncut section may be large enough to retain the support member in the array body and small enough to allow for easy removal of the support member from the array body with breaking the support member. As a non-limiting example, the uncut section may comprise less than 20% of the length of the perimeter. In some embodiments, the uncut section may include less than 10% of the length of the perimeter, for example, from about 0.5% to about 5%. The support member outer perimeter may be circular or non-circular in shape. The support member outer perimeter may include one or more tabs extending outward from the support member body.

The sheet of material may be a thermoplastic. The sheet of material may be cut using a laser cutter or another suitable cutting technique.

Covering the holes with a porous material may include coating a face of the sheet with the porous material. The face may be an upper face or a lower face. The holes are cut in the sheet prior to coating the sheet with the porous material. The outer perimeter of the support member bodies may be cut before or after the sheet is coated with the porous material. If the porous material is applied after the support member outer perimeters are cut, the outer perimeters may be re-cut to cut through the porous material. If the porous material is applied before cutting the outer perimeters, cutting the outer perimeters comprises cutting through the sheet of material and the porous material.

Porous material may be molded in place in the support member bodies. Porous materials may be attached with adhesive and/or bonding.

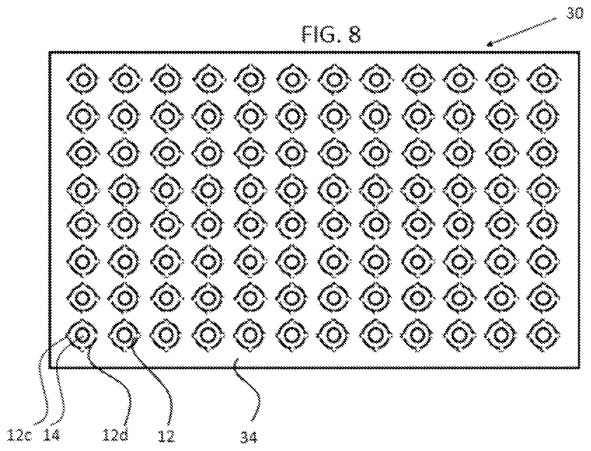

One example showing a partially prepared array 30 of cell support members is shown in FIG. 8, wherein holes 14 and outer perimeters 12d of support member bodies 12 have been cut in a sheet of material 34. In this example, the outer perimeters are substantially circular with tabs 12c. The porous material has not yet been applied.

Forming cell support layers as a sheet comprising an array of cell support members allows for cell support members to be engaged with cell support structures simultaneously, providing high throughput systems.

Cell systems may be prepared to accommodate cell support layers including framed polymer fiber sheets. Cell systems may include cell culture surfaces that include cell culture wells that support the use of reversibly seated framed cell support layers. In some embodiments, framed cell support layers may include those described in United States Publication Number US 2020/0248116, the contents of which are incorporated herein by reference in their entirety, e.g., see pages 7 and 27, and FIGS. 2A-2E of the corresponding application as originally filed).

In some embodiments, cell support layers and polymer fiber components thereof may be prepared according to methods described in the art [e.g., see United States Publication Numbers US 2020/0248116, US 2018/327770 (e.g., see pages 7-10 of the application as originally filed), and US 2009/0317852 (e.g., see paragraph 93 thereof) and U.S. Pat. No. 9,410,267 (e.g., see pages 1-2 of the application as originally filed), the contents of each of which are herein incorporated by reference in their entireties].

### Cell Support Layer Alignment

In some embodiments, the present disclosure provides methods of aligning cell support layers with cell separation systems having cell separation members, including pockets (e.g., microwells) and protrusions (e.g., pillars).

The methods may include using an alignment component to position an array of cell support members with respect to a cell separation system to align individual cell support members with respective separation structures.

The methods may include using a securing component that secures the alignment of the cell support layers with respect to the cell separation system. The securing component may be the same component as the alignment component (e.g., projections that insert into recesses), or a different component (e.g., a clamping mechanism).

The alignment component may include structures on the cell support layer and/or the cell separation system. For example, the cell support layer and the cell separation system may have at least partly the same outer dimensions, such that aligning at least a section of the outer edges of the two components aligns cell support members with respective separation structures. The alignment structures may include one or more projections (e.g., dowels, tabs, shoulders, edges) and corresponding recesses (e.g., holes, slots, grooves) on the cell support layer and the cell separation system that can be fit together to align the support members with respective separation structures.

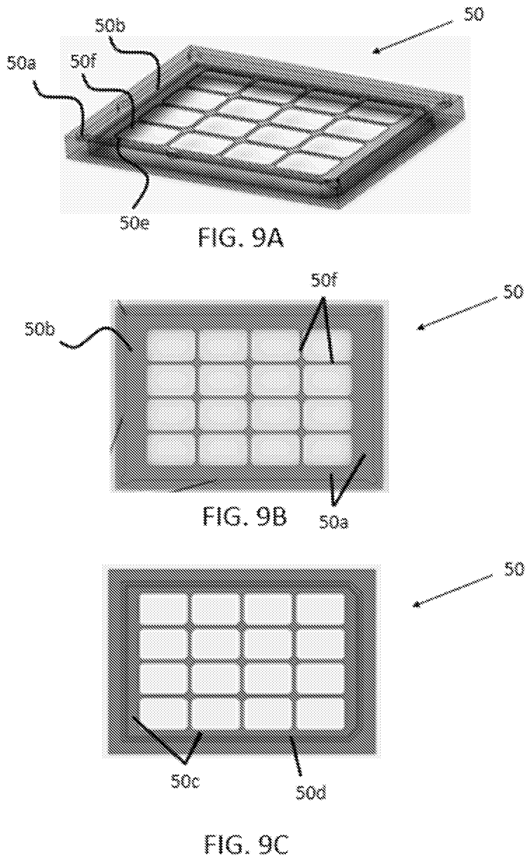

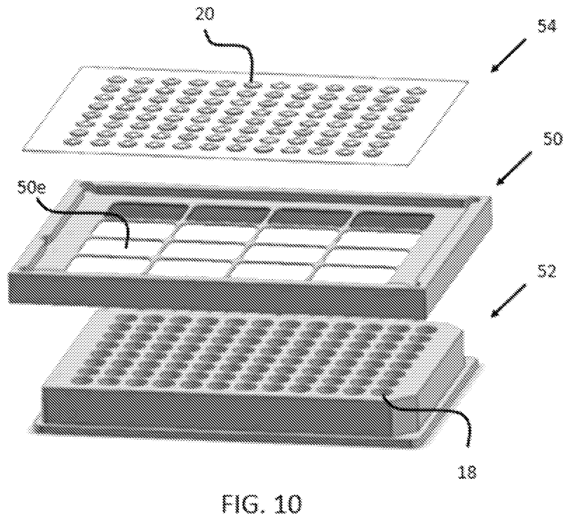

The alignment component may comprise a separate alignment member that can engage with the cell support layer and the cell separation system to align the cell support members with respective separation structures. The alignment member may be a frame having one or more structural features that engage with one or more structural features on the cell support layer and/or the cell separation system to cause alignment of the cell support members and the cell separation structures. The structural features may include one or more projections (e.g., dowels, tabs, shoulders, edges) and corresponding recesses (e.g., holes, slots, grooves). One example of an alignment frame 50 is shown in FIGS. 9A, 9B, 9C, wherein the frame has an upper recess 50a on a top surface 50b for aligning with an outer edge of a cell support layer, and a lower recess 50c on a bottom surface 50d for aligning with an outer edge of a cell separation system. In this example, the frame includes openings 50e that allow support members to be inserted into cell separation system through the openings, and a support grid 50f for supporting the cell separation system on top of the frame. FIG. 10 illustrates how the cell separation system 52 (e.g., with a plurality of wells 18), frame 50 and cell support layer 54 (e.g., an array of support members 20) are layered together, with openings 50e allowing for the support members 20 to be aligned with the wells 18 through the frame 50.

In some embodiments, the frame is configured to be placed on top of the cell support layer that is on top of the cell separation system, causing the alignment of the cell support layer with the cell separation system.

Use of an alignment component to simultaneously align multiple cell support members in an array with multiple cell separation structures allows for high throughput configurations of cell systems.

### Cell Support Layers Placement

In some embodiments, the present disclosure provides methods of inserting cell support members into or over cell separation systems, which may include cell separation features. In some aspects, the cell separation features may include cell separation structures such as pockets, including microwells and/or an array of protrusions.

The methods may comprise aligning a cell support layer, that include an array of cell support members engaged with an array body, with a cell separation system such that cell support members are aligned with respective pockets in the cell separation system or relative to an array of cell separation systems on a vertical plane; imparting a force on the cell support members to disengage cell support members from the array body and insert cell support members into respective pockets or over an array of cell separation systems. The cell support members may be engaged with the array body by a segment of material connecting the cell support members to the array body, and disengaging the cell support members may include separating the segment of material. Cell support members may be disengaged sequentially or simultaneously. Cell support members may be disengaged one at a time or in multiples, including disengaging a subsection of the array or the entire array.

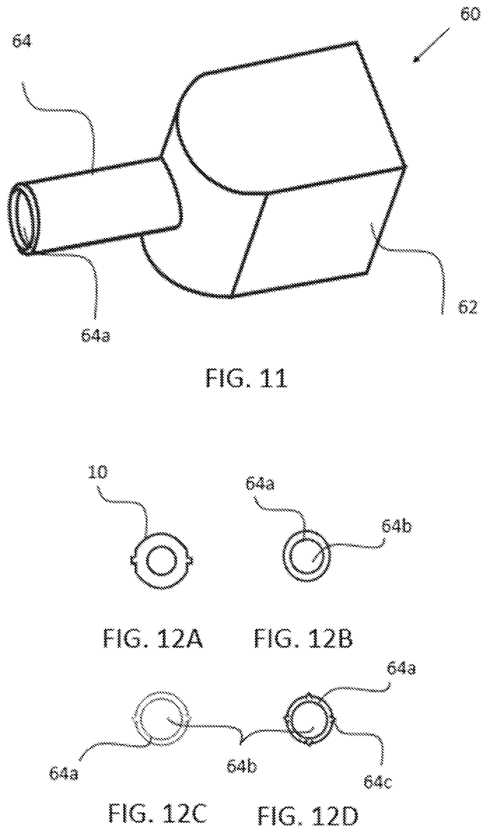

The methods may use a disengagement system (also referred to as a placement system) for applying force to the support members to disengage them from the array body. The disengagement system may be a punch comprising one or more punch heads. FIG. 11 shows one example of a punch 60 having a punch body 62 with a punch head 64 projecting from the punch body and having a distal face 64a. The punch may be used to apply a force (e.g., a downward directed pushing force) to the support member by contacting the distal face of the punch head with an upper face of the support member, thereby disengaging the support member from the array body and forcing it into the pocket of the cell separation system below. The distal face of the punch head may be of suitable dimensions and shape to suitably disengage the support member body. The distal face may have a circular or non-circular shape, and may have one or more tabs extending from the outer perimeter to match tabs in the support member body. FIG. 12A illustrates an example of a support member 10, with suitable punch head distal faces 64a illustrated in FIGS. 12B-D, including punch tabs 64c in FIGS. 12C, 12D. The distal face may have a recess 64b to prevent contact of the punch head with the hole 14 covered by porous material in the support member.

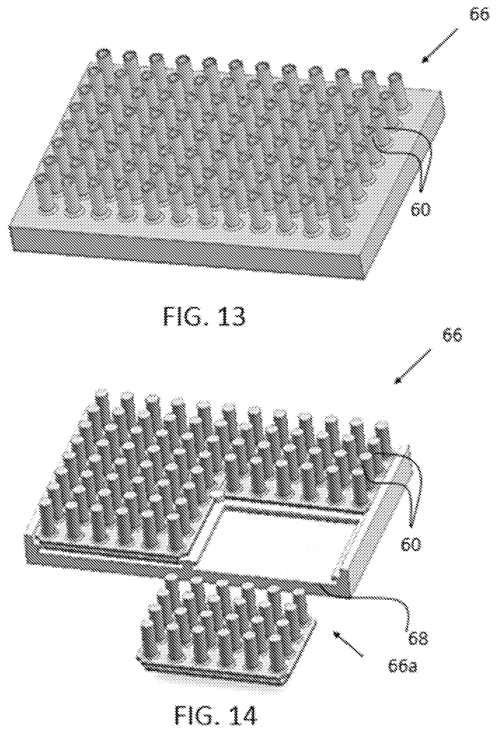

In some embodiments, the disengagement system includes one punch that is mechanically moved over the cell support layer to serially dislodge support members in an array. In some embodiments, the disengagement system includes an array of punches that are spaced apart to align with multiple cell support layers and are actuated together to simultaneously dislodge multiple cell support layers, which may include a portion of the cell support members in the array, or all the cell support members in the array. FIG. 13 shows one example of a disengagement system comprising an array 66 of punches 60. FIG. 14 illustrates one example of a disengagement system comprising an array 66 of punches 60 formed by modular subarrays 66a that fit together on a base 68 to form the array. The punches in an array or subarray may have the same or different design and compositions.

In some embodiments, the modular subarrays may include recesses (e.g. grooves) that may fit within projections extending from the base. Proper alignment of the modular subarrays may be ensured by a projection situated in the center of the array. Each modular subarray may include a particular format or may be formed from a different material.

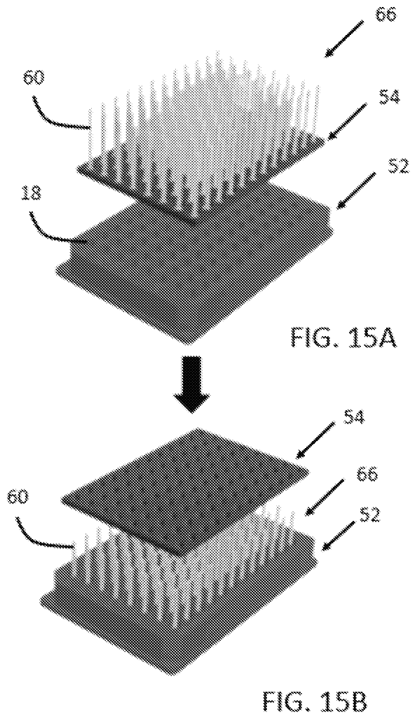

FIGS. 15A, B illustrates one example of a disengagement system comprising an array 66 of punches 60 that are initially positioned above cell support layer 54 (comprising an array of support members) and cell separation system 52 comprising wells 18 as shown in FIG. 15A. The punches are forced through the support layer 54 to dislodge cell support members from the support layer and push them into the pockets, as shown in FIG. 15B.

The punches may be comprised of one or more of plastic, including polylactic acid (PLA), metal, and composite materials. The punches may be manufactured using one or more of milling, molding, lathing and 3D printing. In some embodiments, punches may be coated with materials to prevent the retention of cell culture media and/or porous materials on the surface of the punches.

The disengagement system may include an actuation system for moving the one or more punches and the cell support layer toward one another to apply a force to disengage one or more cell support members. The actuation system may move the one or more punches towards the cell support layer and/or the cell support layer toward the one or more punches. Additional components of the cell system may be moved in conjunction with the cell support layer, including a cell separation system and/or an alignment member. The actuation system may include one or more of manual, automated, or semi-automated. The actuation system may include one or more motors and/or pistons for moving the punches and cell support layer towards each other. The actuation system may be spring-loaded or covered with pliable tip material with the purpose of distributing the force of the punch onto the cell support layer. A pliable material (e.g. sheet of rubber) may also be placed underneath the cell systems to help distribute force of the punch.

In some embodiments, the disengagement system comprises one of more of a pressurized gas system, an articulated member, a vacuum system and a magnetic system for applying force to disengage the support members

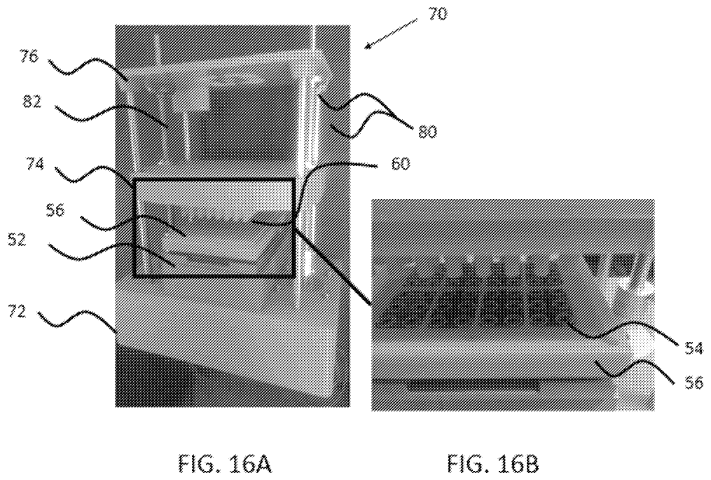

The disengagement system may include an alignment system for aligning punches with cell support members and/or cell separation structures. The alignment system may include alignment members configured to the one or more punches, the cell support layer and the cell separation structures. One example of an alignment system 70 is shown in FIGS. 16A, 16B comprising a base 72 for supporting a cell separation system 52 aligned with a cell support layer 54 comprising an array of support members. A frame 56 is engaged with the cell support layer and includes alignment holes for receiving punches 60 supported on a punch plate 74, with the punches extending downwardly from the punch plate towards the frame and cell support layer. A top plate 76, punch plate 74 and base 72 are connected with smooth rods 80 and threaded rods 82 that feed through smooth holes and threaded holes, respectively, in the punch body and align the punches with the cell support layer 54 and cell separation system 52. The threaded rods are rotated in either direction simultaneously using a dual motor mounted in the base and coupled to the bottom of the threaded rods, which moves the punch body upwards or downwards. Moving the punch plate downwards toward the cell support layer 54 causes the array of punches 60 to apply a force to the support members 10, pushing them into pockets in the cell separation system 52 below. The threaded rods 82 and smooth rods 80 maintain alignment during punching.

In some embodiments, one or more of the punches, cell support layer and cell separation system are mounted on bearings that allow for slight lateral movement of any or all of the layers to align the punches with the cell support members. In some embodiments, the alignment system includes alignment holes for receiving alignment pins and/or punch heads. The alignment holes, alignment pins and/or punch heads may be tapered or chamfered to guide them together during actuation of the disengagement system, thereby aligning the one or more punches with one or more support members. The alignment members may include any one of rails, smooth rods, threaded rods and bearings for aligning punches with cell support members or vice versa. The alignment members may be calibrated or fixed in configuration for multiple cycles or may be mounted on bearings or other low friction elements to allow for slight lateral movement of the layers.

The movement of the disengagement system may be controlled with a microcontroller programmed with functions to allow for prescribed movements of the punches, including movement from a start position to an end position to dislodge the cell support members and movement from the end position back to the start position to reset the disengagement system for another round.

Methods of simultaneously aligning and inserting arrays of cell support members into cell support layers allows for high throughput configurations of cell systems.

### Methods of Analysis & Use

In some embodiments, the present disclosure provides methods of analyzing test factors using cell systems described herein. Such methods may include contacting cell systems (e.g., any of those described herein) with test factors. As used herein the term “test factor” refers to any compound or substance introduced to a cell system for purposes of observing, measuring, or otherwise assessing a cell system response. Methods may include contacting cell systems with control factors, which are factors used for purposes of comparison to test factor effects. Such methods may include analyzing cell system responses to test factors and comparing such responses to cell system responses to control factors.

In some embodiments, methods of the present disclosure include methods of screening for, identifying, and/or testing therapeutic agents. As used herein, the term “therapeutic agent” refers to any substance used to promote the wellbeing of a subject and/or to treat, prevent, alleviate, cure, or diagnose a disease, disorder, or condition. Methods of screening for, identifying, and/or testing therapeutic agents may include contacting cell systems (e.g., any of those described herein) with therapeutic agents or therapeutic agent candidates and measuring at least one outcome associated with such therapeutic agents or candidates. Such methods may further include contacting cell systems with control agents for comparison to therapeutic agents or candidates.

In some embodiments the present disclosure provides methods of preparing cells for various uses. For example, cells, such as adipocytes, may be grown for use in tissue reconstruction. Adipocytes may also be prepared for creating end products like lab grown meat from animal adipocytes.

### III. Definitions

Adjacent: As used herein, the term “adjacent” refers to something that is adjoining, neighboring or next to a given entity, which may or may not be touching or may or may not be spaced apart from the given entity.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 130, 12%, 11%, 100, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions.

Device: As used herein, the term “device” refers to any article constructed or modified to suit a particular purpose.

Distal: As used herein, the term “distal” means situated away from the center or away from a point or region of interest.

Engineered: As used herein, embodiments of the disclosure are “engineered” when they are designed to have a feature or property, whether structural or chemical, that varies from a starting point, wild type, or native molecule. Thus, engineered agents or entities are those whose design and/or production include an act of the hand of man.

Essentially. As used herein, the term “essentially” is used to indicate some degree of variation associated with a basic property or characterization. For example, multiple lengths that vary in size by around 5% may be characterized as being essentially equal.

Feature: As used herein, a “feature” refers to a characteristic, a property, or a distinctive element.

Kit: As used herein, the term “kit” refers to a set that includes one or more components adapted for a cooperative purpose and instructions for use thereof.

Proximal: As used herein, the term “proximal” means situated nearer to the center or to a point or region of interest.

Synthetic: The term “synthetic” means produced, prepared, and/or manufactured by the hand of man. Synthesis of polymers or other molecules of the present disclosure may include chemical or enzymatic synthesis.

### IV. Equivalents and Scope

While various disclosure embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The disclosure includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Microwell Cell System

A cell system is prepared to include an arrangement of individual cells. The cell system includes a cell culture surface with a pattern of etched microwells with an extracellular matrix coating within each microwell. The microwells are patterned to facilitate spacing of individual cell system cells. A low-density solution of cells is prepared and added to the culture surface to facilitate cell attachment to the coated microwells before unattached cells are removed. A cell support member inserted into or over each microwell to prevent cells from floating and detaching from cell attachment surfaces in the microwells.

### Example 2. High Throughout Microwell Cell System

A cell system is prepared according to the description in Example 1, with the difference that the cell culture surface is part of a 96-well dish (although multi-well dishes with other well multiples may also be used).

### Example 3. Adipocyte Cell Systems

Cell systems according to Example 1 or 2 are prepared for preparation, maintenance, and analysis of mature adipocytes. With each system, preadipocytes are used to prepare low density cell solutions used to introduce cells. Spacing between the microwells facilitates preadipocyte maturation and hypertrophy, while cell support layers maintain attachment of the cells to the cell culture surface.

What is claimed is:

1. A cell separation system comprising:
(i) a substrate; and
(ii) a cell separation feature in or on the substrate for spatially separating individual cells.

2. The cell separation system of claim 1, wherein the cell separation feature provides a cell spacing between adjacent cells of from about 5 μm to about 500 μm.

3. The cell separation system of claim 1 or 2, wherein the substrate comprises a cell culture surface.

4. The cell separation system of claim 3, wherein the cell culture surface comprises one or more of a cell culture dish, a cell culture well, a plastic, a resin, an elastomer, a metal and glass.

5. The cell separation system of any one of claims 1-4, wherein the cell separation feature comprises any one of or combination of a cell separation structure and a coating layer for promoting or discouraging cell attachment.

6. The cell separation system of any one of claims 1-5, wherein the cell separation structure comprises a pocket in the cell culture surface, and the pocket is preferably a microwell.

7. The cell separation system of any one of claims 1-5, wherein the cell separation structure comprises a projection from the cell culture surface, and the projection is preferably a pillar.

8. The cell separation system of any one of claims 1-7, wherein the cell separation feature provides a cell attachment surface having one or more of a diameter of from about from about 25 μm to about 50 μm and a cross sectional area of from about 400 μm² to about 200 μm².

9. The cell separation system of claim 8, wherein the cell attachment surface is substantially flat.

10. The cell separation system of claim 8 or 9, wherein the cell attachment surface is circular, oblong, rectangular, or irregular in shape.

11. The cell separation system of any one of claims 8-10, wherein the cell separation feature comprises a pocket, and the cell attachment surface comprises a bottom surface of the pocket.

12. The cell separation system of any one of claims 8-10, wherein the cell separation structure comprises a projection, and the cell attachment surface comprises a top surface of the projection.

13. The cell separation system of any one of claims 1-12, wherein the cell separation feature is formed by one or more of etching, micromolding, milling and 3D printing.

14. The cell separation system of any one of claims 1-12, further comprising a coating layer.

15. The cell separation system of any one of claims 1-14, wherein the cell separation structure provides a cell attachment surface, and the coating layer covers the cell attachment surface.

16. The cell separation system of claim 14 or 15, wherein the coating layer comprises a coating protein.

17. The cell separation system of claim 16, wherein the coating protein comprises an extracellular matrix protein.

18. The cell separation system of claim 17, wherein the extracellular matrix protein comprises collagen, laminin, fibronectin, gelatin, or a fragment or combination thereof.

19. The cell separation system of any one of claims 1-18, comprising an array of the cell separation structures in or on the substrate.

20. A cell support member comprising a porous material for maintaining a position of a cell on a substrate, the cell support member configured for placement over the cell to prevent passage of the cell through the porous material while allowing fluid communication through the porous material.

21. The cell support member of claim 20, the cell support member further comprising:
(i) a cell support body; and
(ii) at least one hole through the cell support body for allowing fluid communication through the support body, wherein the porous material covers the at least one hole.

22. The cell support member of claim 21, wherein the cell support body comprises a thermoplastic material.

23. The cell support member of any one of claims 21-22, wherein the porous material covers at least one face of the cell support body.

24. The cell support member of claim 20, wherein the porous material is a membrane or a disc.

25. The cell support member of claim 21 or 22, wherein the porous material comprises a synthetic polymer and/or a biogenic polymer.

26. The cell support member of any one of claims 20-25, wherein the porous material comprises a synthetic polymer, and wherein the synthetic polymer comprises one or more of a poly(urethane), a poly(siloxane), a poly(ethylene), a poly(vinyl pyrrolidone), a poly(2-hydroxy ethyl methacrylate), a poly(N-vinyl pyrrolidone), a poly(methyl methacrylate), a poly(vinyl alcohol), a poly(acrylic acid), a polyacrylamide, a poly(ethylene-co-vinyl acetate), a poly(ethylene glycol), a poly(methacrylic acid), a polylactides (PLA), a polyglycolide (PGA), a poly(lactide-co-glycolide) (PLGA), a polyanhydride, a polyphosphazene, a polygermane, a polyorthoester, a polyester, a polyamide, a polyolefin, a polycarbonate, a polyaramide, a polyimide, a polycaprolactone (PCL), and a copolymer, derivative, or combination thereof.

27. The cell support member of any one of claims 20-26, wherein the porous material comprises a biogenic polymer, and wherein the biogenic polymer comprises one or more of a protein, a polypeptide, a polysaccharide, a lipid, a nucleic acid, a glycosaminoglycan, a derivative, or combination thereof.

28. The cell support member of any one of claims 20-27, wherein the porous material comprises a biogenic polymer, and wherein the biogenic polymer comprises one or more of silk, fibroin, sericin, keratin, alpha-keratin, beta-keratin, alginate, elastin, fibrillin, fibrillin-1, fibrillin-2, fibrillin-3, fibrillin-4, fibrinogen, fibrin, fibronectin, laminin, collagen, collagen I, collagen II, collagen III, collagen IV, collagen V, collagen VI, vimentin, neurofilament, light chain neurofilament (NF-L), medium chain neurofilament (NF-M), heavy chain neurofilament (NF-H), amyloid, alpha-amyloid, beta-amyloid, actin, myosin, titin, gelatin, chitin, hyaluronic acid, D-glucuronic acid, D-N-acetylglucosamine, a derivative, or a combination thereof.

29. The cell support member of any one of claims 20-28, wherein the porous material comprises one or more polymer fibers.

30. The cell support member of claim 29, wherein the polymer fibers have a diameter of from about 10 nm to about 5000 nm.

31. The cell support member of claim 30, wherein the polymer fibers have a diameter of from about 200 nm to about 1000 nm.

32. The cell support member of any one of claims 29-31, wherein the polymer fibers comprise PCL.

33. The cell support member of any one of claims 29-32, wherein the polymer fibers comprise gelatin.

34. The cell support member of claim 32 or 33, wherein the polymer fibers comprise at least about 50 wt % PCL.

35. The cell support member of any one of claims 29-34, wherein the polymer fibers comprise from about 1 to about 49 wt % gelatin.

36. The cell support member of any one of claims 31-35, wherein the polymer fibers comprise about 70 wt % PCL and about 30 wt % gelatin.

37. The cell support member of any one of claims 20-36, wherein the porous material is formed from one or a combination of controlled spinning of nanofibers, lithography, deposition, etching, molding and crystal growth.

38. The cell support member of any one of claims 20-37, wherein the porous material has a porosity of from about 20% to about 50%.

39. The cell support member of any one of claims 20-37, wherein the average pore size in the porous material is from about 1 μm to about 5 μm.

40. The cell support member of any one of claims 20-39, wherein the cell support member is substantially planar.

41. The cell support member of any one of claims 20-40, wherein the cell support member is substantially tubular.

42. The cell support member of any one of claims 20-41, wherein the cell support member has one or more tabs projecting from its outer perimeter for engaging with a cell separation system for aligning and/or anchoring the cell support member to the cell separation system.

43. A cell support layer comprising an array of cell support members as defined in any one of claims 20-42, wherein the cell support members are retained by an array body.

44. A cell system comprising:
(i) a cell separation system as defined in any one of claims 1-19; and
(ii) a cell support member as defined in any one of claims 20-42, configured for engagement with the cell separation structure.

45. A cell system comprising:
(i) the array of cell separation structures as defined in claim 19; and
(ii) the cell support layer of claim 43, wherein the array of cell support members are configured to align with the array of cell separation structures.

46. The cell system of claim 44 or 45, wherein the cell support member comprises a substantially planar cell support body configured for insertion into or over the cell separation structure comprising pockets or placement over a projection of the cell separation structure.

47. The cell system of claim 44 or 45, wherein the cell support member comprises a substantially tubular cell support body configured for insertion into or over the cell separation structure comprising pockets or placement over a projection of the cell separation structure.

48. The cell system of any one of claims 44-47, comprising an arrangement of individual cells, said individual cells comprising progenitor cells.

49. The cell system of claim 48, wherein the progenitor cells comprise mesenchymal progenitor cells.

50. The cell system of any one of claims 44-49, wherein the arrangement of individual cells comprises one or more of a pre-adipocyte and a mature adipocyte.

51. The cell system of claim 50, wherein the mature adipocyte comprises a diameter of from about 15 μm to about 400 μm.

52. The cell system of any one of claims 44-51, wherein the distance between adjacent cells is from about 5 μm to about 500 μm.

53. The cell system of any one of claims 44-51, wherein the cell system is configured for high throughput analysis of at least one test factor.

54. The cell system of claim 53, wherein the cell system is configured for analysis of multiple conditions relative to the at least one test factor.

55. A placement system for engaging a cell support member with a cell separation system comprising:
(i) a punch having at least one punch head with a distal face;
(ii) an actuating system for engaging the at least one punch head with the cell support member to disengage the cell support member from a cell support layer, and to engage the cell support member with the cell separation system; and
(iii) optionally, an alignment system for aligning the punch with the cell support member.

56. The placement system of claim 55, wherein the cell support member is defined in any one of claims 20-42 and the cell separation system is defined in any one of claims 1-19.

57. The placement system of claim 56, wherein the punch comprises a plurality of punch heads configured for alignment with an array of cell support members on the cell support layer as defined in claim 43, for disengaging multiple cell support members from the cell support layer simultaneously.

58. The placement system of any one of claims 55-57, wherein the at least one punch head comprises a recess on its distal face for preventing contact between the distal face and at least one hole in the cell support member during disengagement of the cell support member.

59. The placement system of any one of claims 55-58, wherein the actuating system is actuated manually, automatically or semi-automatically.

60. The placement system of any one of claims 55-58, wherein the actuating system comprises any one or combination of one or more motors, pulleys and pistons.

61. A method of forming a cell support member comprising the steps of:
(i) providing a sheet of material;
(ii) cutting at least one hole in the sheet of material;
(iii) forming a support member body from the sheet of material by cutting an outer perimeter of the support member body around the hole; and
(iv) applying a porous material over the hole.

62. The method of claim 61, wherein the cell support member is defined in any one of claims 19-41.

63. The method of claim 61 or 62, wherein when cutting the outer perimeter of the support member body, at least a portion of the outer perimeter is left uncut such that the support member body remains connected to the sheet of material.

64. The method of any one of claims 61-63, where multiple support members are formed in the sheet of material to form an array of support members.

65. The method of any one of claims 61-64, wherein the porous material is applied over one face of the sheet of material, covering the holes and the support member bodies.

66. A method of preparing a cell system, the method comprising:
(i) providing a cell separation system comprising a cell separation structure on a substrate; and
(ii) associating a cell with an attachment area on the substrate.

67. The method of claim 66, wherein the cell separation system is defined in any one of claims 1-18.

68. The method of claim 66 or 67, further comprising the step of applying a coating layer to the attachment area.

69. The method of claim 68, wherein the coating layer comprises a coating protein.

70. The method of claim 69, wherein the coating protein comprises an extracellular matrix protein.

71. The method of claim 70, wherein the extracellular matrix protein comprises collagen, laminin, fibronectin, gelatin, or a fragment of combination thereof.

72. The method of any one of claims 68-71, wherein applying the coating layer comprises microcontact printing, photolabile polymer printing, and/or stamping.

73. The method of any one of claims 68-72, wherein the coating layer is applied to the attachment area by positioning a blocking mask over the substrate, wherein the blocking mask comprises a hole and wherein the blocking mask is positioned to align the hole with the attachment area; applying the coating layer to the blocking mask; and removing the blocking mask to yield application of the coating layer to the attachment area.

74. The method of any one of claims 68-73, wherein from about 0.01 μg to about 50 μg of the coating protein are applied per square centimeter of attachment area.

75. The method of any one of claims 68-74, wherein associating the cell with the attachment area comprises contacting the coating layer with a solution of cells, allowing the cell to associate with the coating layer, and removing any cells not associated with the coating layer.

76. The method of claim 75, wherein the solution of cells comprises a cell concentration suitable to provide from about 1 to about 10,000 cells per square millimeter of attachment area.

77. The method of any one of claims 66-76, wherein the cell comprises one or more of a progenitor cell, a mesenchymal progenitor cell, a pre-adipocyte, and a mature adipocyte.

78. The method of any one of claims 66-77, further comprising the step of:
(i) covering the cell with a cell support member after the cell is associated with the attachment area.

79. The method of claim 78, wherein the cell support member is defined in any one of claims 20-43.

80. The method of claim 78 or 79, wherein the cell support member is inserted into or over a pocket of the cell separation system.

81. The method of any one of claims 78-80, wherein the cell support member is placed over a projection of the cell separation system.

82. The method of any one of claims 78-81, wherein the cell support member is formed according to the method of any one of claims 61-65.

83. The method of any one of claims 78-82, wherein covering the cell with the cell support member comprises:
(i) providing a cell support layer comprising an array of cell support members retained by an array body;
(ii) aligning the cell support layer over the cell separation system such that the cell support members are aligned on a vertical plane with respective cell separation structures; and
(iii) applying a force to the cell support members to disengage them from the array body and engage them with the cell separation structures.

84. The method of claim 83, wherein all the cell support members in the array are disengaged simultaneously.

85. The method of claim 83 or 84, wherein the array of cell support members is formed according to the method of claim 64.

86. The method of claim 84 or 85, wherein the placement system of any one of claims 54-60 is used to disengage the cell support members.

87. The method of any one of claims 83-86, wherein an alignment system is present and is used to align the cell support layer over the cell separation system.

88. The method of claim 87, wherein the alignment system comprises a frame configured for aligning with structural features on the cell support layer and the cell separation system.

89. The method of claim 87, wherein the alignment system comprises one or more projections and one or more corresponding recesses on any one or combination of the cell support layer, the cell separation system, and a separate alignment member.

90. The method of any one of claims 66-89, wherein the cell system is configured for high throughput analysis of at least one test factor.

91. The method of claim 90, wherein the cell system is configured for analysis of multiple conditions relative to the at least one test factor.

92. A method of analyzing a test factor, the method comprising contacting the cell system of any one of claims 43-54 with the test factor.

93. The method of claim 92 comprising contacting the cell system with a control factor and analyzing the test factor by comparison to the control factor.

94. A method of screening for, identifying, or testing a therapeutic agent, the method comprising contacting the cell system of any one of claims 43-54 with the therapeutic agent and measuring at least one outcome associated with the therapeutic agent.

95. The method of claim 94, further comprising contacting the cell system with a control agent, measuring at least one outcome associated with the control agent, and comparing the outcome associated with the control agent with the outcome associated with the therapeutic agent.

96. A device for assembly of a cell system according any one of claims 44-54.

97. The device of claim 96, comprising at least one reservoir layer, wherein the reservoir layer is positioned above the cell separation system.

98. The device of claim 97, comprising the cell support layer, wherein the reservoir layer is positioned above the cell support layer.

99. The device of any one of claims 96-98, wherein the cell support layer is positioned above the cell separation system.

100. The device of claim 98, comprising a gasket between the reservoir layer and the cell support layer.

101. The device of claim 100, comprising a gasket between the cell support layer and a base layer.
